(19) United States
(12) Patent Application Publication
(10) Pub. No.: US 2019/0190445 A1
(43) Pub. Date: Jun. 20, 2019

(54) DIAGNOSTIC VEHICLES FOR MAINTAINING SOLAR COLLECTOR SYSTEMS

(71) Applicant: Alion Energy, Inc., Richmond, CA (US)

(72) Inventors: Jason Alderman, Duarte, CA (US); Luigi Petrigh-Dove, Petaluma, CA (US); Brett West, Concord, CA (US); Craig Wildman, Kensington, CA (US)

(21) Appl. No.: 16/201,905

(22) Filed: Nov. 27, 2018

Related U.S. Application Data
(60) Provisional application No. 62/591,644, filed on Nov. 28, 2017.

(51) Int. Cl. H02S 50/15; H02S 20/32; G01N 17/00; G01J 1/42; G01N 33/00

(52) U.S. Cl. CPC H02S 50/15; H02S 20/32; G01J 2001/4266; G01J 1/4204; G01N 33/0098; G01N 17/006

(57) ABSTRACT

Diagnostic vehicles, systems, and methods for characterizing a solar collector system are presented herein. The diagnostic vehicle comprises a frame, one or more sensors positioned along the frame, and a control system. The one or more sensors measure and characterize attributes of the solar collector system and/or its environment such as reflectivity of an area of ground around the solar collector system, an angular offset of a drive system of the solar collector system, and/or a degradation of structural components that support photovoltaic panels in the solar collector system. The control system is programmed to move the frame to one or more locations in the solar collector system and control the one or more sensors to acquire measurements at the one or more locations.

### CROSS-REFERENCE TO RELATED APPLICATION

This application claims priority to U.S. Provisional Application No. 62/591,644, filed Nov. 28, 2017, the entirety of which is herein incorporated by reference.

### FIELD

This disclosure pertains to solar photovoltaic (PV) power plants.

### BACKGROUND

Photovoltaic panels have a front side and a back side and have in the past typically only collected light from the front side. The equipment designed to work with such monofacial photovoltaic panels has been designed to accommodate panels that receive light only from the front. Similarly, operation and maintenance processes have been designed for panels that only collect light from the front.

Some photovoltaic panels, known as bifacial panels, can collect light from both the front side and the back side. Embodiments described herein apply to solar collectors both monofacial and bifacial panels.

### SUMMARY

A diagnostic vehicle for characterizing a solar collector system is presented herein. The diagnostic vehicle comprises a frame, one or more sensors positioned along the frame, and a control system. The one or more sensors measure and characterize attributes of the solar collector system and/or its environment such as a reflectivity of an area of ground around the solar collector system, an angular offset of a drive system of the solar collector system, and/or a degradation of structural components that support photovoltaic panels in the solar collector system. The control system is programmed to move the frame to one or more locations in the solar collector system and control the one or more sensors to acquire measurements at the one or more locations.

A method for characterizing a solar collector system is presented herein. In the method, one or more sensors positioned along a frame measure at least one of a reflectivity of an area of ground around the solar collector system, an angular offset of a drive system of the solar collector system, or a degradation of structural components that support photovoltaic panels in the solar collector system. A control system moves the frame to one or more locations in the solar collector system. The control system controls the one or more sensors to acquire measurements at the one or more locations.

A system that comprises a solar collector system and a diagnostic vehicle that traverses the solar collector system is presented herein. The diagnostic vehicle comprises a vehicle frame, one or more sensors positioned along the frame to measure and characterize a reflectivity of an area of ground around the solar collector system, and an applicator configured to distribute, based on the reflectivity, a reflective material on an area of ground around the solar collector system. The diagnostic vehicle further comprises a control system programmed to move the frame to one or more locations in the solar collector system and control the one or more sensors to acquire measurements at the one or more locations.

### BRIEF DESCRIPTION OF THE FIGURES

FIG. 1 schematically illustrates a perspective view of an embodiment of a diagnostic vehicle on a solar collector.

FIG. 2 shows a side view of a diagnostic vehicle on a solar collector.

FIG. 3 depicts a diagnostics vehicle with a camera positioned to observe a backside of solar panels, such as to observe a bar code on the panel.

FIG. 4 shows a side view of a diagnostic vehicle on a solar collector positioned to observe solar collector structure, solar collector foundation, nearby ground, and vegetation growth.

FIG. 5 depicts a diagnostic vehicle that can be used to measure degradation in a drive system of a solar tracker.

FIG. 6 depicts a diagnostic vehicle positioned under a solar collector.

FIG. 7 depicts another view of a diagnostic vehicle positioned under a solar collector.

FIG. 8 depicts a flow diagram for characterizing a solar collector system.

### DETAILED DESCRIPTION

Solar collectors that use either monofacial panels or bifacial panels face issues of quality of installation, wear and degradation, electrical issues, plant growth around the structure, and a myriad of other problems. Diagnostics can be performed to improve decision making for plant managers, such as by helping to decide when to do maintenance activities. Unfortunately, making measurements or observations of solar panels can be very expensive and yet can also provide a much smaller sample size of observations than is desired. A crew takes time to drive far into the countryside to get to a solar plant and then has time to check only a small number panels or other equipment out of thousands on a typical utility-scale solar plant. Plant managers are left paying significant expenses without getting the information they desire to make good decisions.

Measurements and diagnostics can help managers of a solar plant in a variety of ways such as by identifying and characterizing problems, by optimizing plant performance, by observing the state of components for wear or degradation, or by simply observing what components are in which locations in a solar power plant. Measurements and diagnostics are important for solar plants with both monofacial panels and bifacial panels, and the following systems and methods apply to both kinds.

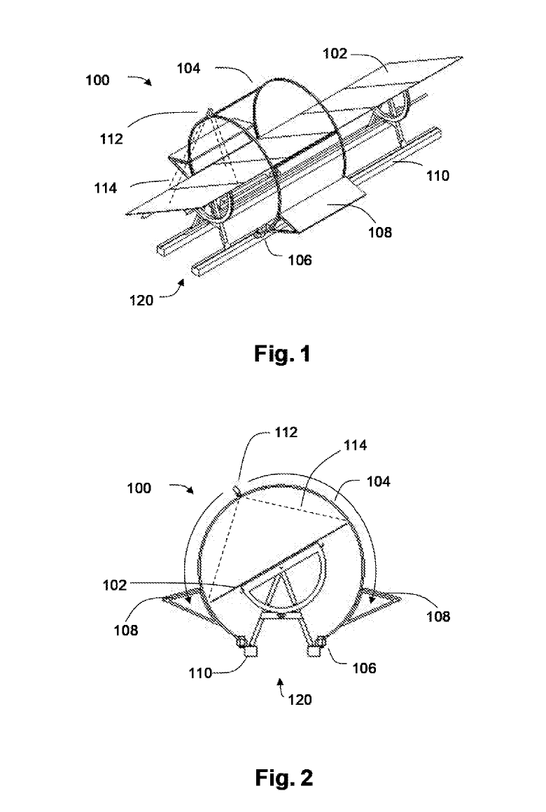

FIG. 1 schematically illustrates a perspective view of an embodiment of a diagnostic vehicle 100 on a solar collector 120. The diagnostic vehicle 100 includes a structure 104 on four sets of wheels 106, two sets on each side of the solar collector. Each of the four wheel sets 106 consists of a weight-bearing wheel that rides on the top of the solar collector's concrete ballast 110 or other track and also a guide wheel that rolls along the sides of the concrete track. One or more of the weight-bearing wheels 106 can be a driving wheel that is powered directly or via a transmission by an electric motor or combustion engine. The diagnostic vehicle 100 can also be pulled or pushed by workers, by another vehicle, by a cable, or by other means. The diagnostic vehicle 100 can be powered by one or more solar panels 102 which can charge an onboard battery to store energy. The diagnostic vehicle 100 can alternatively be powered by a battery that is occasionally electrically connected to a charger to recharge the battery or swapped out for a fresh battery. Alternatively, the diagnostic vehicle 100 can be powered by a combustion engine with a fuel tank, can be powered manually, by other means, or by a combination of the above methods.

The diagnostic vehicle 100 can include a control system, memory, and a wireless communication system as well. The diagnostic vehicle 100 can be remotely programmed and directed to drive to one or more specific locations, to acquire images and store them as data, and to transmit the data to other computer systems. The diagnostic vehicle 100 also can be equipped with means of determining its position via GPS, via using RFID reader and RFID tags that are placed in its path, via using optical observations of its surroundings, by proximity sensors interacting with corresponding targets, or by other means. The diagnostic vehicle 100 can have a variety of other components too, such as implements for a maintenance process, storage of consumables, an onboard pretreatment system to treat consumables before depositing them, or other components or systems.

Continuing with FIG. 1, the diagnostic vehicle 100 can carry one or more cameras or other optical sensors 112. The cameras 112 can take images of solar panels 102 in the infrared (IR) spectrum, in the visible spectrum, or in other sections of the electromagnetic spectrum. Multiple cameras 112 can take multiple images at once. A solar panel typically comprises a number of solar cells. The vehicle frame 104 is configured to position the camera 112 sufficiently far away from the solar panels 102 so a number of cells fits in the camera's field of view at the same time. The camera 112 can also be angled to look down the row of solar panels 102 to further increase the number of cells or panels that fits inside the camera's field of view. The extent of an example field of view is diagrammed with dashed lines 114.

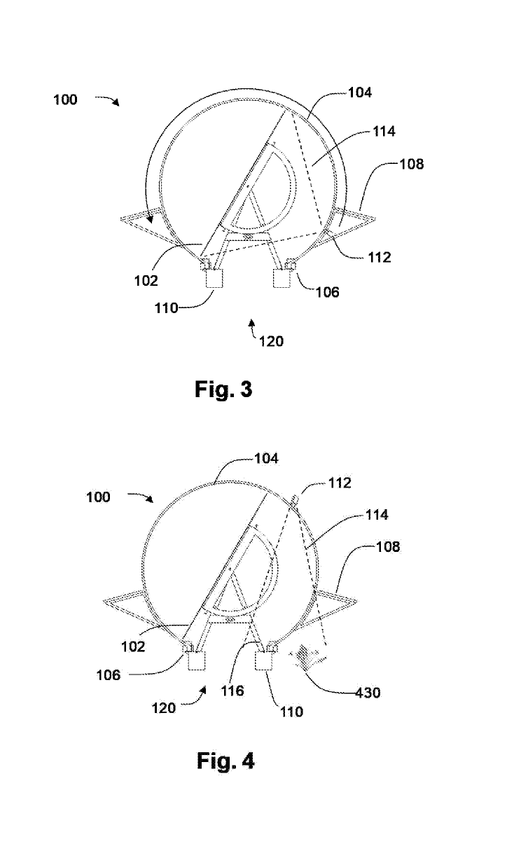

FIG. 2 shows a side view of the diagnostic vehicle 100 on a solar collector 120. The vehicle frame 104 can be shaped like a circle in the direction of this view, and the azimuthal position of the camera 112 around the frame can be adjusted so that the camera 112 can be positioned anywhere along this circle. If the diagnostic vehicle 100 is operating on a fixed-tilt solar collector, the camera 112 can be set in a single position, such as aligned with the normal vector of the solar panels 102, to take images of the solar collector. If the diagnostic vehicle 100 is operating on a tracking solar collector, as is shown in FIGS. 1 and 2, the camera's azimuthal position on the vehicle frame 104 can be continually or intermittently adjusted to account for the movement of the solar panels 102. The camera 112 can be moved by an electric motor with a chain or belt transmission, by manual repositioning, by pneumatics, or by other means. As shown in FIG. 3, the camera can also be positioned to capture images of the rear of the panels or positioned to capture whatever other images are desired.

The camera 112 can be an IR camera, a visible-spectrum (normal) camera, or a combination of both. Infrared spectrum images of operating solar panels can be helpful to show cell temperature, variations of temperature within a cell, and variations of temperature between cells. Temperature measurements can help an operator deduce problems with electrical performance. Combining an IR image with a visible-spectrum image can further aid an operator in understanding solar cell and solar panel performance. With such a camera tool, this diagnostic vehicle 100 can be used to drive along a row of operating solar collectors and collect images in the IR and visible spectra. A computer program can then be used to process the images where results can include flagging anomalies for further attention or generating statistics on a large batch of samples. In this way, an operator can program the diagnostic vehicle 100 to drive through an operating solar plant, capture a number of IR and visible images, to store the data, to process the data on board, and to transmit the data to another computer for further processing, storage, and investigation by the operator. This system and method would be a substantial improvement over other methods of recording images. It would be much cheaper than sending a person to walk around the field with a camera. Besides cost, many solar plants are impractically large to pay people to capture images of all of the panels. Another advantage of the diagnostic vehicle 100 is that the camera is held still and at a uniform position, thus improving image quality and easing image processing. As opposed to aerial vehicles that carry cameras, this land-based vehicle can stop and become motionless to take a picture. This can increase the flexibility in the photography process, for example by allowing a longer exposure duration without blurring the picture.

In an alternative embodiment, the camera 112 in FIG. 3 can be a bar code reader or matrix bar code reader and can be positioned to observe the backside of the panels 102 at the position where the panel's bar codes are positioned. In this embodiment, the diagnostic vehicle 100 can drive along a row of solar collectors and capture images of panel bar codes. Combining the bar code data with the sequence of when pictures were taken or with the vehicle position can enable a computer program to make a map or catalogue of a solar field of thousands of solar panels to show which solar panels are in which locations. This can be helpful for diagnostic purposes because, for example, electrical problems identified via IR imaging or by other means can be matched to manufacturing batches of panels. Furthermore, construction crews typically do not record panel identification and location when installing them.

In an alternative embodiment such as diagrammed in FIG. 3, the camera 112 can take visible-spectrum or IR images of any structural members or electrical connections behind the solar panels 112. These images can be processed to identify construction quality issues, wear marks on the bearing surfaces, other degradation issues, or other problems. For example, images of all of the electrical connections in a solar field can be taken and processed after construction to check that they were all made correctly. Images of all of the metal parts of a solar field can be taken and processed from time to time to check for corrosion. Images of drive system components can be taken and processed to check for wear or increased gaps at bearing surfaces or for other signs of degradation.

FIG. 4 shows a side view of a diagnostic vehicle 100 where the camera 112 is positioned on the vehicle's frame 104 and is rotated on its mount such that its field of view (denoted by dashed lines 114) includes the solar collector's concrete track 110 or other foundation, the solar collector's mounting structure 108, the connection between mounting structure 108 and the foundation, and the area of ground around the concrete track 110. In this configuration, the diagnostic vehicle 100 can be used to drive along and capture images of the foundation, track, structure, and connections between foundation and structure. This can be used to check for quality or defects during installation, such as of an epoxy joint between the concrete and support structure. It can also be used to check for concrete track wear, concrete cracking, track deterioration, and for metal corrosion. It can further be used to check for debris or obstructions on the vehicle track 110. By capturing images either in IR or visible spectrum of the ground near the solar collector and with subsequent image processing, the diagnostic vehicle 100 can be used to survey how much plant growth 430 has occurred and to help determine whether cutting the plants is necessary. This can also be useful for surveying for other issues like snow accumulation, water pooling, settlement of the ground, animal activity, or other natural phenomena. Conducting surveys with the diagnostic vehicle 100 and subsequent image processing can result in significant cost savings over sending work crews to site to go out and walk the site to check for issues.

In FIG. 4, the camera 112 can alternatively be a pyranometer, radiometer, or other sensor to measure light. It is possible to deposit agricultural lime or other material to increase the reflectivity of the ground, also called its albedo. By increasing the ground's reflectivity, the solar panels 102, and especially bifacial panels, will receive more light reflected from the ground. However, natural phenomena, like rain, can deteriorate the reflectivity benefit of such a ground treatment. A diagnostic vehicle 100 with a sensor to measure light oriented downward at the ground can be used to measure the albedo to help a plant manager decide whether to reapply the treatment that increases albedo.

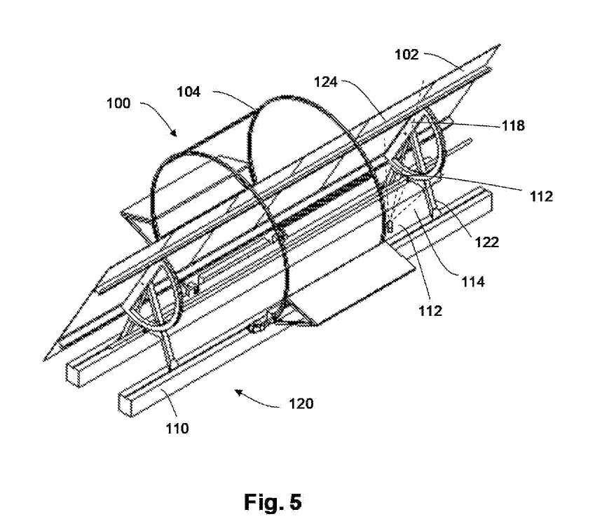

Another way to use the diagnostic vehicle 100 is to measure a degradation in a drive system of the solar tracker 120, as diagrammed in FIG. 5. The solar tracker 120 can use a single drive motor coupled to a long drive shaft or torque tube to provide power and torque to a number of solar tracker sections or a single tracker section, as shown in FIG. 5. The torque tube or drive shaft is not perfectly stiff. Torsional flexibility can arise from mechanical fastener tolerances at connection points in the drive shaft, from inherent flexibility in the material, or from other causes. Over time the stiffness of the solar tracker 120 can reduce with wear.

In FIG. 5, the solar panels 102 are supported by two purlins 124. The purlins 124 are connected by two pivot arms 118, which are perpendicular to the purlins 124. The pivot arms 118 rotate about an axle at the top of a support structure 122. The support structure 122 is supported by the ballast foundation 110.

A method to measure the flexibility of a tracker row is as follows. The diagnostic vehicle 100 can be positioned at a tracker section of the row furthest from the drive motor. The diagnostic vehicle 100 can be positioned so that the camera's field of view 114 includes the rotating structural components of the solar tracker 120, such as the pivot arm 118, relative to the structural components 122 that are fixed to the foundation. The drive motor can rotate all of the tracker sections to their furthest rotational position away from the diagnostic vehicle's camera 112. Then, the motor can slowly rotate the tracker back toward horizontal. The angle of the motor's encoder can be compared with the angle observed at the last tracker section observed by the camera 112 to estimate the twist in the drive shaft or torque tube between the motor and the last tracker section. Image processing can be used to compare the relative angle of the rotational components such as the pivot arm 118 with the stationary structural components 122 to calculate the flexibility of the drive system.

A solar tracker 120 can have a stow position to address strong wind conditions. The stow position can be designed so that the tracker structure can be made much stronger, much stiffer, or better protected from wind forces, or so that the drive system can be decoupled. The diagnostic vehicle 100 of FIG. 5 can be used to verify on a regular basis that all of the tracker sections 120 of a solar plant properly enter and exit the stow position. The camera 112 can be used to capture images of the drive components or other critical components. Then, imaging software can be used to process the data and to flag those few tracker sections that are not performing properly. Because a large solar plant can have thousands of tracker sections, such verification would otherwise be prohibitively expensive.

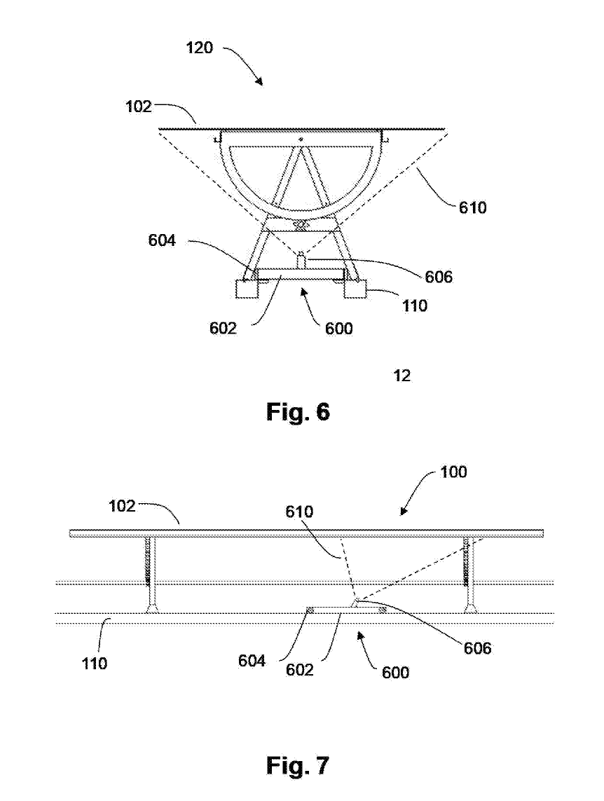

In FIGS. 6 and 7, an alternative diagnostic vehicle 600 is shown positioned under a solar collector 120. The diagnostic vehicle includes a body 602 which can be carried by four wheel sets 604. A wheel set 604 can include a weight-bearing wheel which rolls on the top of the solar collector's concrete track 110 and a guiding wheel that rolls along the inside of the concrete track 110. The diagnostic vehicle 600 can be solar powered with battery energy storage, powered with a battery and plug-in charger, powered with a battery that can be swapped out for a fresh battery, powered by an engine, or powered by other means such as manually or by a cable. The diagnostic vehicle 600 has a control system, memory, and a communication system as well. The diagnostic vehicle 600 can be programmed and directed remotely to drive to one or more specific locations, to acquire images and store them as data, and to transmit the data to other computer systems. The diagnostic vehicle 600 also has means of determining its position by GPS, by using RFID reader and RFID tags, by using optical observations of its surroundings, or by other means.

Continuing with FIGS. 6 and 7, the diagnostic vehicle 600 also includes a camera 606. The camera 606 can be positioned to point upward at the solar panels 102, support structure, and drive system of the solar collector 120. The camera 606 can be oriented to look down the line of solar panels 102 to increase the field of view (denoted with dashed lines 610) so that the entire width of the solar panels 102 is in view. An angular position of the camera 606 can be changed by onboard motors to observe different view of the solar collector 120, for example to keep the solar panels 102 in view as they track the sun. The camera 606 can be an IR camera that observes the solar cells for temperature, a visible-spectrum camera and an IR camera, or a visible-spectrum camera. The camera 606 can be directed and focused to observe the solar panels 102, structural members of the solar collector, or drive system members of the solar collector. The diagnostic vehicle 100 can take images during an operation of the solar collector 120 or when the solar collector 120 is not operating. This diagnostic vehicle embodiment can be used to observe tracker section movement, driveshaft twist, and other mechanical behavior in the same methods as the diagnostic vehicle of FIGS. 1-7.

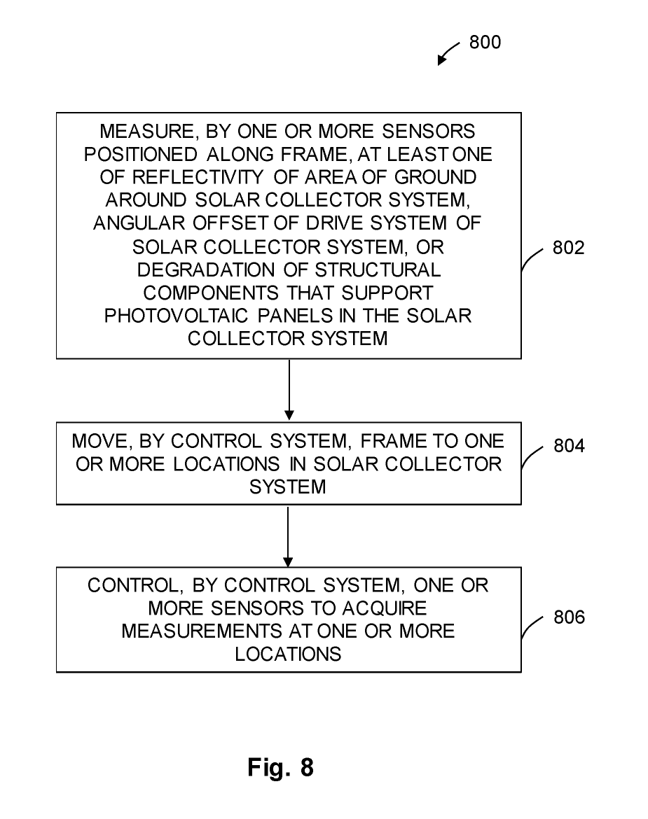

FIG. 8 depicts a flow diagram for characterizing a solar collector system. At 802, one or more sensors positioned along a frame measure at least one of a reflectivity of an area of ground around the solar collector system, an angular offset of a drive system of the solar collector system, or a degradation of structural components that support photovoltaic panels in the solar collector system. A control system moves the frame to one or more locations in the solar collector system at 804. At 806, the control system controls the one or more sensors to acquire measurements at the one or more locations.

In the descriptions above and in the claims, phrases such as “at least one of” or “one or more of” can occur followed by a conjunctive list of elements or features. The term “and/or” can also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A diagnostic vehicle for characterizing a solar collector system, the diagnostic vehicle comprising:
a frame;
one or more sensors positioned along the frame to measure and characterize at least one of a reflectivity of an area of ground around the solar collector system, an angular offset of a drive system of the solar collector system, or a degradation of structural components that support photovoltaic panels in the solar collector system; and
a control system programmed to move the frame to one or more locations in the solar collector system and control the one or more sensors to acquire measurements at the one or more locations.

2. The diagnostic vehicle of claim 1, wherein the frame circumferentially extends around photovoltaic panels in the solar collector system.

3. The diagnostic vehicle of claim 2, wherein at least one of the one or more sensors are configured to move along a circumference of the frame.

4. The diagnostic vehicle of claim 1, wherein the angular offset of the drive system of the solar collector system is combined with information from a motor to estimate a degradation in the drive system.

5. The diagnostic vehicle of claim 1, wherein the solar collector system further comprises:
a row of photovoltaic panels; and
a support structure to support and rotate the row of photovoltaic panels, wherein the support structure comprises metal, and the one or more sensors characterize and measure a level of corrosion of one or more structural components of the support structure.

6. The diagnostic vehicle of claim 1, wherein the one or more sensors comprise at least one of a pyranometer, a radiometer, or a photometer.

7. The diagnostic vehicle of claim 1, the diagnostic vehicle further comprising:
a wireless communications system that transmits data characterizing the measurements at the one or more locations to a remote computing system for analysis.

8. The diagnostic vehicle of claim 1, the diagnostic vehicle further comprising:
an applicator for applying a reflective material to the area of ground to increase the reflectivity of the area of ground based on an analysis of the measurements.

9. The diagnostic vehicle of claim 1, wherein the one or more sensors comprise a camera that captures images of vegetation growth on the area of ground.

10. The diagnostic vehicle of claim 1, wherein an azimuthal position of the one or more sensors are adjusted by the control system to account for a movement of the row of photovoltaic panels.

11. The diagnostic vehicle of claim 1, wherein the one or more sensors measure a temperature of the solar collector system to diagnose or characterize an electrical performance of the solar collector system.

12. The diagnostic vehicle of claim 5, wherein the diagnostic vehicle further comprises:
a bar code reader positioned to capture bar codes on a backside of the row of photovoltaic panels, wherein the bar codes are combined with the measurements at the one or more locations to generate a mapping that includes an identification of at least one photovoltaic panels, a location of the at least one photovoltaic panel, and at least one measurement corresponding to the at least one photovoltaic panel from the measurements at the one or more locations.

13. The diagnostic vehicle of claim 1, the solar collector system further comprising:
rotating components;
a drive motor programmed with a desired angle of rotation of the photovoltaic panels;
wherein the angular offset is measured by positioning the diagnostic vehicle at a location away from the drive motor, positioning the one or more sensors to measure an actual angle of rotation of the rotating components of the solar collector system, and comparing the desired angle with the actual angle.

14. A method for characterizing a solar collector system, the method comprising:
measuring, by one or more sensors positioned along a frame, at least one of a reflectivity of an area of ground around the solar collector system, an angular offset of a drive system of the solar collector system, or a degradation of structural components that support photovoltaic panels in the solar collector system;
moving, by a control system, the frame to one or more locations in the solar collector system; and
controlling, by the control system, the one or more sensors to acquire measurements at the one or more locations.

15. The method of claim 14, wherein the frame circumferentially extends around photovoltaic panels in the solar collector system.

16. The method of claim 15, wherein the sensor is configured to move along a circumference of the frame.

17. The method of claim 14, wherein the angular offset of the drive system of the solar collector system is combined with information from a motor to estimate a degradation in the drive system.

18. The method of claim 14, wherein the solar collector system further comprises:
a row of photovoltaic panels; and
a support structure to support and rotate the row of photovoltaic panels, wherein the support structure comprises metal, and the one or more sensors characterize and measure a level of corrosion of one or more structural components of the support structure.

19. The method of claim 14, wherein the one or more sensors comprise at least one of a pyranometer, a radiometer, or a photometer.

20. The method of claim 14, the method further comprising:
transmitting, by a wireless communications system, data comprising the measurements at the one or more locations to a remote computing system for analysis.

21. The method of claim 14, the method further comprising:
applying, by an applicator, a reflective material to the area of ground to increase the reflectivity of the area of ground based on an analysis of the measurements.

22. The method of claim 14, wherein the one or more sensors comprise a camera that captures images of vegetation growth on the area of ground.

23. The method of claim 14, wherein an azimuthal position of the one or more sensors are adjusted by the control system to account for a movement of the row of photovoltaic panels.

24. The method of claim 14, wherein the one or more sensors measure a temperature of the solar collector system to diagnose or characterize an electrical performance of the solar collector system.

25. The method of claim 18, wherein the method further comprises:
positioning a bar code reader to capture bar codes on a backside of the row of photovoltaic panels, wherein the bar codes are combined with the measurements at the one or more locations to generate a mapping that includes an identification of at least one photovoltaic panels, a location of the at least one photovoltaic panel, and at least one measurement corresponding to the at least one photovoltaic panel from the measurements at the one or more locations.

26. The method of claim 14, the solar collector system further comprising:
rotating components;
a drive motor programmed with a desired angle of rotation of the photovoltaic panels;
wherein the angular offset is measured by positioning the diagnostic vehicle at a location away from the drive motor, positioning the one or more sensors to measure an actual angle of rotation of the rotating components of the solar collector system, and comparing the desired angle with the actual angle.

27. A system comprising:
a solar collector system;
a diagnostic vehicle that traverses the solar collector system, the diagnostic vehicle comprising:
a vehicle frame;
one or more sensors positioned along the frame to measure and characterize a reflectivity of an area of ground around the solar collector system
an applicator configured to distribute, based on the reflectivity, a reflective material on an area of ground around the solar collector system; and
a control system programmed to move the frame to one or more locations in the solar collector system and control the one or more sensors to acquire measurements at the one or more locations.
